(19) United States
(12) Patent Application Publication
(10) Pub. No.: US 2010/0111887 A1
(43) Pub. Date: May 6, 2010

(54) COMPOSITION WITH A HEATING EFFECT CONTAINING FOAMING SURFACTANTS

(75) Inventors: Jerome SENEE, Lardy (FR); Delphine Compain, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/430,247

(22) Filed: Apr. 27, 2009

Related U.S. Application Data
(60) Provisional application No. 61/050,638, filed on May 6, 2008.

(30) Foreign Application Priority Data
Apr. 25, 2008 (FR) 08 52811

(51) Int. Cl. A61K 8/19; A61Q 1/14; A61Q 19/10; A61Q 9/02

(52) U.S. Cl. 424/73; 510/136; 510/137

(57) ABSTRACT

Composition containing, in an anhydrous medium, at least one exothermic metal salt, at least a first foaming surfactant and at least a second foaming surfactant, the composition containing less than 1% of gelling agent.

### REFERENCE TO PRIOR APPLICATIONS

This application claims priority to U.S. provisional application Ser. No. 61/050,638, filed May 6, 2008; and to French patent application 08 52811, filed Apr. 25, 2008, both incorporated herein by reference.

### FIELD OF THE INVENTION

The subject of the present invention is an anhydrous composition comprising a metal salt, and at least two foaming surfactants. Preferably, the composition comprises less than 1% of gelling agent.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

It is known to use anhydrous cosmetic compositions that are exothermic, that is to say that have a heating effect during application to the skin. The heating products may contain various raw materials which, in the presence of water, release heat. As raw materials of this type, there may be mentioned for example polyols such as glycerine and glycols, salts such as calcium chloride or magnesium sulphate, or zeolites.

Polyols alone give a heat effect which is low. The salts or the zeolites are efficient alone or in combination with the polyols, but it is nevertheless necessary to incorporate into the compositions a high percentage of salts (for example 10 to 30%) in order to have a satisfactory heat effect, that is to say an increase of 10° C. to 30° C. during humidification by addition of water or during application to wet skin. Unfortunately, salts are known to destabilize cosmetic compositions, and the presence of gelling agents such as for example hydrophilic or hydrophobic silicas is then necessary to stabilize the compositions.

### SUMMARY OF THE INVENTION

The inventors have found, surprisingly, that it is possible to formulate compositions, including cosmetic compositions, with a heating effect comprising a salt, which are stable and which contain little or no gelling agent, by including therein at least two foaming surfactants. Preferably the at least two foaming surfactants are provided in a specific ratio.

### DETAILED DESCRIPTION OF THE PREFERRED EMBODIMENTS

As used herein, the words “a” and “an” and the like carry the meaning of “one or more.”

In one embodiment the invention relates to a composition, preferably a cosmetic composition (all compositions described herein are preferably cosmetic compositions, but this is not a requirement) comprising, in an anhydrous medium, an exothermic metal salt, an amphoteric foaming surfactant and an anionic foaming surfactant, preferably in an anionic surfactant/amphoteric surfactant ratio ranging from 0.5 to 2, the composition preferably comprising less than 1% of gelling agent.

The composition of the invention is exothermic, that is to say that it has a heating effect and therefore that the user feels heating during the application of the composition to wet or moistened skin. It is a composition whose temperature in the presence of water (the water added during use or alternatively the water present in the skin) can increase by several degrees (one to twenty degrees) essentially instantly, or instantly. This heating effect will allow the opening of the pores of the skin and therefore better cleansing of the skin.

The composition according to the invention preferably uses an anhydrous medium. In a preferred embodiment the entire composition is anhydrous. The expression “anhydrous” is understood here to mean a practically anhydrous medium, that is to say generally comprising less than 6% by weight of water, preferably less than 4% by weight of water, and better still less than 1% by weight of water relative to the total weight of the composition. Both the medium and the entire composition are each preferably completely anhydrous and therefore free of water.

The composition of the invention preferably being a cosmetic composition and therefore intended for topical application, it preferably comprises a physiologically acceptable medium, that is to say compatible with the skin, the mucous membranes and/or keratin fibres.

Foaming Surfactants

The amphoteric surfactant may be chosen for example from betaines, N-alkylamidobetaines and their derivatives, sultaines, alkyl polyaminocarboxylates, alkyl amphoacetates and mixtures thereof.

1) As betaines, there may be mentioned in particular alkylbetaines such as for example cocobetaine such as the product marketed under the name DEHYTON AB-30® by the company Cognis, laurylbetaine such as the product marketed under the name GENAGEN KB® by the company Clariant, oxyethylenated (10 EO) laurylbetaine, such as the product marketed under the name LAURYLETHER (10 EO) BETAINE® by the company Shin Nihon Rica, oxyethylenated stearylbetaine (10 EO) such as the product marketed under the name STEARYLETHER (10 EO) BETAINE® by the company Shin Nihon Rica.

Among the N-alkylamidobetaines and their derivatives, there may be mentioned for example cocamidopropylbetaine marketed under the name LEBON 2000 HG® by the company Sanyo, or marketed under the name EMPIGEN BB® by the company Albright & Wilson, lauramidopropylbetaine marketed under the name REWOTERIC AMB12P® by the company Witco.

2) As sultaines, there may be mentioned hydroxylsultaines, such as cocamidopropyl-hydroxysultaine such as the product marketed under the name REWOTERIC AM CAS by the company Golschmidt-Degussa, or the product marketed under the name CROSULTAINE C-50® by the company Croda.

3) As alkyl polyaminocarboxylates (APAC), there may be mentioned sodium cocoyl polyaminocarboxylate marketed under the name AMPHOLAK 7 CX/C® and AMPHOLAK 7 CX® by the company Akzo Nobel, sodium stearyl polyamidocarboxylate marketed under the name AMPHOLAK 7 TX/C by the company Akzo Nobel, sodium carboxymethyloleylpolypropylamine marketed under the name AMPHOLAK XO7/C® by the company Akzo Nobel.

4) As alkyl amphoacetates, there may be mentioned for example N-disodium N-cocoyl-N-carboxymethoxyethyl-N-carboxymethylethylenediamine (INCI name: disodium cocoamphodiacetate) such as the product marketed under the name MIRANOL C2M CONCENTRE NP® by the company Rhodia, N-sodium N-cocoyl-N-hydroxyethyl-N-carboxymethylethylenediamine (INCI name: sodium cocamphoacetate), sodium cocoamphohydroxypropyl sulphonate marketed under the name MIRANOL CSE by the company Rhodia.

The anionic foaming surfactant(s) may be chosen in particular from the anionic derivatives of proteins of plant origin or of silk proteins, phosphates and alkyl phosphates, carboxylates, sulphosuccinates, amino acid derivatives, alkyl sulphates, alkyl ether sulphates, sulphonates, isethionates, taurates, alkyl sulphoacetates, polypeptides, the anionic derivatives of alkyl polyglucoside, and mixtures thereof.

1) The anionic derivatives of proteins of plant origin are hydrolysates of protein with a hydrophobic group, it being possible for the hydrophobic group to be naturally present in the protein or to be added by the reaction of the protein and/or the protein hydrolysate with a hydrophobic compound. The proteins are of plant origin or are derived from silk, and the hydrophobic group may be in particular a fatty chain, for example an alkyl chain comprising from 10 to 22 carbon atoms. As anionic derivatives of proteins of plant origin, there may be mentioned more particularly the hydrolysates of apple, wheat, soybean or oat proteins comprising an alkyl chain having from 10 to 22 carbon atoms and their salts. The alkyl chain may be in particular a lauryl chain and the salt may be a sodium, potassium and/or ammonium salt.

Accordingly, as protein hydrolysates with a hydrophobic group, there may be mentioned for example the salts of the hydrolysates of silk protein modified with lauric acid, such as the product marketed under the name KAWA SILK by the company Kawaken; the salts of the hydrolysates of wheat protein modified with lauric acid, such as the potassium salt marketed under the name Aminofoam W OR by the company Croda (INCI name: Potassium lauroyl wheat aminoacids) and the sodium salt marketed under the name PROTEOL LW 30 by the company Seppic (INCI name: sodium lauroyl wheat aminoacids); the salts of the hydrolysates of oat protein comprising an alkyl chain having from 10 to 22 carbon atoms, and more especially the salts of the hydrolysates of oat protein modified with lauric acid, such as the sodium salt marketed under the name PROTEOL OAT (30% aqueous solution) by the company Seppic (INCI name: Sodium lauroyl oat aminoacids); the salts of the hydrolysates of apple protein, comprising an alkyl chain having from 10 to 22 carbon atoms, such as the sodium salt marketed under the name PROTEOL APL (30% hydroglycolic solution) by the company Seppic (INCI name: Sodium Cocoyl Apple amino acids). Mention may also be made of the mixture of lauroyl amino acids (aspartic acid, glutamic acid, glycine, alanine) neutralized with sodium N-methylglycinate, marketed under the name PROTEOL SAV 50 S by the company Seppic (INCI name: Sodium Cocoyl amino acids).

2) As phosphates and alkyl phosphates, mention may be made for example of the monoalkyl phosphates and the dialkyl phosphates, such as the lauryl monophosphate marketed under the name MAP 20® by the company Kao Chemicals, the potassium salt of dodecylphosphoric acid, mixture of mono- or diester (diester predominantly) marketed under the name CRAFOL AP-31® by the company Cognis, the mixture of monoester and diester of octylphosphoric acid marketed under the name CRAFOL AP-20® by the company Cognis, the mixture of ethoxylated (7 moles of EO) phosphoric acid monoester and diester of 2-butyloctanol, marketed under the name ISOFOL 12 7 EO-PHOSPHATE ESTER® by the company Condea, the potassium or triethanolamine salt of (C₁₂-C₁₃) monoalkyl phosphate marketed under the references ARLATONE MAP 230K-40® and ARLATONE MAP 230T-60® by the company Uniqema, potassium lauryl phosphate marketed under the name DERMALCARE MAP XC-99/09® by the company Rhodia Chimie, and potassium cetylphosphate marketed under the name ARLATONE MAP 160K by the company Uniqema.

3) As carboxylates, there may be mentioned:
The amidoether carboxylates (AEC), such as sodium lauryl amidoether carboxylate (3 EO) marketed under the name AKYPO FOAM 30® by the company Kao Chemicals.
The polyoxyethylenated carboxylic acid salts, such as the oxyethylenated (6 EO) sodium lauryl ether carboxylate (C₁₂₋₁₄₋₁₆ 65/25/10) marketed under the name AKYPO SOFT 45 NV® by the company Kao Chemicals, the polyoxyethylenated and carboxymethylated fatty acids of olive oil origin marketed under the name OLIVEM 400® by the company BIOLOGIA E TECNOLOGIA, the oxyethylenated (6 EO) sodium tridecyl ether carboxylate marketed under the name NIKKOL ECTD-6NEX® by the company Nikkol.
The salts of fatty acids having a C₆ to C₂₂ alkyl chain, neutralized with an organic or inorganic base, which constitute soaps. The fatty acid salt or soap is obtained from a fatty acid and a base, the fatty acid comprising a saturated or unsaturated, linear or branched alkyl chain having from 12 to 22 carbon atoms and preferably 12 to 20 carbon atoms. The bases (also called saponification agents) completely or partially neutralize the fatty acids. The bases which may be used to obtain the salts may be for example inorganic bases such as alkali metal hydroxides (sodium hydroxide and potassium hydroxide), alkaline-earth metal hydroxides (magnesium hydroxide) or ammonium hydroxide, or alternatively organic bases such as triethanolamine, N-methylglucamine, lysine and arginine. The fatty acid may be chosen in particular from C₁₀ to C₂₄, and in particular C₁₂-C₁₈, fatty acids, and in particular lauric acid, myristic acid, stearic acid, palmitic acid and mixtures thereof.

4) As amino acid derivatives, there may be mentioned in particular the alkali metal salts of amino acids, such as:
sarcosinates, such as sodium lauroyl sarcosinate marketed under the name SARKOSYL NL 97® by the company Ciba or marketed under the name ORAMIX L 30® by the company Seppic, sodium myristoyl sarcosinate marketed under the name NIKKOL SARCOSINATE MN® by the company Nikkol, sodium palmitoyl sarcosinate marketed under the name NIKKOL SARCOSINATE PN® by the company Nikkol;
alaninates, such as sodium N-lauroyl-N-methyl amidopropionate marketed under the name SODIUM NIKKOL ALANINATE LN 30® by the company Nikkol, or marketed under the name ALANONE ALE® by the company Kawaken, N-lauroyl-N-methylalanine triethanolamine marketed under the name ALANONE ALTA® by the company Kawaken;
glutamates, such as triethanolamine monococoyl glutamate marketed under the name ACYLGLUTAMATE CT-12® by the company Ajinomoto, triethanolamine lauroyl glutamate marketed under the name ACYLGLUTAMATE LT-12® by the company Ajinomoto;
aspartates, such as the mixture of triethanolamine N-lauroyl aspartate/triethanolamine N-myristoyl aspartate marketed under the name ASPARACK® by the company Mitsubishi;
glycine derivatives (glycinates), such as sodium N-cocoyl glycinate marketed under the names AMILITE GCS-12® and AMILITE GCK 12 by the company Ajinomoto;
citrates such as oxyethylenated citric monoester of coconut alcohols (9 moles), marketed under the name WITCONOL EC 1129 by the company Goldschmidt;
galacturonates such as sodium dodecyl D-galactoside uronate marketed by the company Soliance.

5) As sulphosuccinates, there may be mentioned for example oxyethylenated (3 EO) lauryl alcohol monosulphosuccinate (C₁₂/C₁₄ 70/30) marketed under the names SETACIN 103 SPECIAL®, REWOPOL SB-FA 30 K 4® by the company Witco, the disodium salt of a hemisulphosuccinate of C₁₂-C₁₄ alcohols marketed under the name SETACIN F SPECIAL PASTE® by the company Zschimmer Schwarz, oxyethylenated (2 EO) disodium oleamidosulphosuccinate marketed under the name STANDAPOL SH 135® by the company Cognis, oxyethylenated (5 EO) lauryl amide monosulphosuccinate marketed under the name LEBON A-5000® by the company Sanyo, the oxyethylenated (10 EO) disodium salt of lauryl citrate monosulphosuccinate marketed under the name REWOPOL SB CS 50® by the company Witco, ricinoleic monoethanolamide monosulphosuccinate marketed under the name REWODERM S 1333® by the company Witco. It is also possible to use polydimethylsiloxane sulphosuccinates such as disodium PEG-12 dimethicone sulphosuccinate marketed under the name MACKANATE-DC30 by the company Mac Intyre.

6) As alkyl sulphates, there may be mentioned for example triethanolamine lauryl sulphate (INCI name: TEA-lauryl sulfate) such as the product marketed by the company Huntsman under the name EMPICOL TL40 FL or that marketed by the company Cognis under the name TEXAPON T42, which products are in aqueous solution at 40%. Mention may also be made of ammonium lauryl sulphate (CFTA name: Ammonium lauryl sulfate) such as the product marketed by the company Huntsman under the name EMPICOL AL 30FL which is in aqueous solution at 30%.

7) As alkyl ether sulphates, there may be mentioned for example sodium lauryl ether sulphate (INCI name: sodium laureth sulfate) such as that marketed under the names TEXAPON N40 and TEXAPON AOS 225 UP by the company Cognis or such as that marketed under the name EMPICOL ESB 3/FL3 by the company Huntsman, ammonium lauryl ether sulphate (INCI name: ammonium laureth sulfate) such as that marketed under the name STANDAPOL EA-2 by the company Cognis.

8) As sulphonates, there may be mentioned for example alpha-olefin sulphonates such as sodium (C₁₄₋₁₆) alpha-olefin sulphonate marketed under the name BIO-TERGE AS-40® by the company Stepan, marketed under the names WITCONATE AOS PROTEGE® and SULFRAMINE AOS PH 12® 12 by the company Witco or marketed under the name BIO-TERGE AS-40 CG® by the company Stepan, the secondary sodium olefin sulphonate marketed under the name HOSTAPUR SAS 30® by the company Clariant; linear alkyl aryl sulphonates such as sodium xylene sulphonate marketed under the names MANROSOL SXS30®, MANROSOL SXS40®, MANROSOL SXS93® by the company Manro.

9) As isethionates, there may be mentioned acyl isethionates such as sodium cocoyl isethionate, such as the product marketed under the name JORDAPON CI P® by the company Jordan.

10) As taurates, there may be mentioned the sodium salt of palm kernel oil methyl taurate marketed under the name HOSTAPON CT PATE® by the company Clariant; N-acyl N-methyl taurates such as sodium N-cocoyl N-methyl taurate marketed under the name HOSTAPON LT-SF® by the company Clariant or marketed under the name NIKKOL CMT-30-T® by the company Nikkol, sodium palmitoyl methyl taurate marketed under the name NIKKOL PMT® by the company Nikkol.

11) The anionic derivatives of alkyl polyglucosides may be in particular glycerol citrates, tartrates, sulphosuccinates, carbonates and ethers obtained from alkyl polyglucosides. There may be mentioned for example the sodium salt of cocoyl polyglucoside (1.4) tartaric ester, marketed under the name EUCAROL AGE-ET® by the company Cesalpinia, the disodium salt of cocoyl polyglucoside (1.4) sulphosuccinic ester, marketed under the name ESSAI 512 MP® by the company Seppic, the sodium salt of cocoyl polyglucoside (1.4) citric ester marketed under the name EUCAROL AGE-EC® by the company Cesalpinia.

Preferably, the amphoteric surfactant is chosen from N-alkylamidobetaines and their derivatives, in particular cocamidopropylbetaine. Also preferably, the anionic surfactant is chosen from alkyl sulphates and their salts, for example sodium lauryl sulphate. Combinations of one or more N-alkylamidobetaines and their derivatives with one or more alkyl sulphates and their salts are also preferred.

Advantageously, the amphoteric surfactant and the anionic surfactant are present in the composition in an anionic surfactant/amphoteric surfactant ratio ranging for example from 0.5 to 2.0, preferably 0.7 to 1.5, for example of the order of 1, including 0.6, 0.8, 0.9, 1.1, 1.2, 1.3, and 1.4, 1.6, 1.7, 1.8 and 1.9 and all values and subranges between stated values.

The composition of the invention may contain additionally to the amphoteric surfactant and the anionic surfactant, at least an additional foaming surfactant that may be chosen for example from non ionic surfactants.

The nonionic foaming surfactants may be chosen in particular from alkyl polyglucosides (APG), oxyalkylenated glycerol esters, oxyalkylenated sugar esters, and mixtures thereof.

1) As alkyl polyglucosides, use is preferably made of those containing an alkyl group comprising from 6 to 30 carbon atoms and preferably from 8 to 16 carbon atoms, and containing a glucoside group preferably comprising 1, 2 to 3 glucoside units. The alkyl polyglucosides may be chosen for example from decyl glucoside (C9/C11 alkyl polyglucoside (1.4)) such as the product marketed under the name Mydol 10® by the company Kao Chemicals or the product marketed under the name Plantacare 2000 UP® by the company Cognis; caprylyl/capryl glucoside such as the product marketed under the name Plantacare KE 3711® by the company Cognis; lauryl glucoside such as the product marketed under the name Plantacare 1200 UP® by the company Cognis; cocoglucoside such as the product marketed under the name Plantacare 818 UP® by the company Cognis; caprylyl glucoside such as the product marketed under the name Plantacare 810 UP® by the company Cognis; and mixtures thereof.

2) The oxyalkylenated glycerol esters are in particular the polyoxyethylenated derivatives of esters of glyceryl and of a fatty acid and of their hydrogenated derivatives. These oxyalkylenated glycerol esters may be chosen for example from hydrogenated and oxyethylenated glyceryl esters of fatty acids such as PEG-200 hydrogenated glyceryl palmate marketed under the name Rewoderm LI-S 80 by the company Goldschmidt; oxyethylenated glyceryl cocoates such as PEG-7 glyceryl cocoate marketed under the name Tegosoft GC by the company Goldschmidt, and PEG-30 glyceryl cocoate marketed under the name Rewoderm LI-63 by the company Goldschmidt; and mixtures thereof.

3) The oxyalkylenated sugar esters are in particular polyethylene glycol ethers of fatty acid esters of sugars. These oxyalkylenated sugar esters may be chosen for example from oxyethylenated glucose esters such as PEG-120 methyl glucose dioleate marketed under the name Glucamate DOE 120 by the company Amerchol.

According to a preferred embodiment of the invention, the nonionic surfactant is an alkyl polyglucoside which may be chosen in particular from decyl glucoside, caprylyl/capryl glucoside, lauryl glucoside, cocoglucoside, caprylyl glucoside, and mixtures thereof.

The foaming surfactants may be present in a total amount ranging for example from 0.1 to 30% by weight relative to the total weight of the composition, preferably from 1 to 15% and even better from 2 to 10% by weight, including for example 3, 5, 7, 9, 11, 13, 17, 19, 21, 23, 25, 27 and 29% including all values and subranges between stated values.

Gelling Agents

The composition of the invention preferably comprises less than 1% gelling agents, preferably less than 0.5% gelling agents, for example preferably less than 0.9, 0.8, 0.7, 0.6, 0.4, 0.3, 0.2 and 0.1%, and better still is free of gelling agents. Preferably, the amount of gelling agent is expressed based on the total weight of the composition.

The expression gelling agent (also called thickening agents) is understood to mean an agent capable of modifying the viscosity of the composition. The fillers and exfoliating particles mentioned later are not considered as gelling agents.

As lipophilic or hydrophilic gelling agents, there may be mentioned:
organophilic clays which are clays modified with chemical compounds which make the clay capable of swelling in oily media; by way of examples of such products, there may be mentioned clays of the smectite family such as montmorillonites, hectorites, bentonites, beidellites, saponites, and of the family comprising vermiculites, stevensite, chlorites.

These clays may be modified with a chemical compound chosen from quaternary amines, tertiary amines, amine acetates, imidazolines, amine soaps, fatty sulphates, alkyl aryl sulphonates, amine oxides, and mixtures thereof;
pyrogenic silicas which may be obtained by high-temperature hydrolysis of a volatile silicon compound in an oxyhydric flame, producing a finely divided silica. This process makes it possible in particular to obtain hydrophilic silicas which have a large number of silanol groups at their surface. Such hydrophilic silicas are for example marketed under the names “AEROSIL 130®”, “AEROSIL 200®”, “AEROSIL 255®”, “AEROSIL 300®”, “AEROSIL 380®” by the company Degussa, “CAB-β-SIL HS-5®”, “CAB-O-SIL EH-5®”, “CAB-O-SIL LM-130®”, “CAB-O-SIL MS-55®”, “CAB-O-SIL M-5®” by the company Cabot.
Silicas modified with hydrophobic groups such as trimethylsiloxyl groups, which are obtained in particular by treating pyrogenic silica in the presence of hexamethyldisilazane. Silicas thus treated are called “Silica silylate” according to the CTFA (6th edition, 1995). They are for example marketed under the references “AEROSIL R812®” by the company Degussa, “CAB-O-SIL TS-530®” by the company Cabot. Dimethylsilyloxyl or polydimethylsiloxane groups, which are in particular obtained by treating pyrogenic silica in the presence of polydimethylsiloxane or dimethyldichlorosilane. Silicas thus treated are called “Silica dimethyl silylate” according to the CTFA (6th edition, 1995). They are for example marketed under the references “AEROSIL R972®”, “AEROSIL R974®” by the company Degussa, “CAB-O-SIL TS-610®”, “CAB-O-SIL TS-720®” by the company Cabot,
the homo- or copolymers of acrylic or methacrylic acids and their salts and their esters and in particular the products sold under the names “VERSICOL F” or “VERSICOL K” by the company ALLIED COLLOID, “UTRAHOLD 8” by the company CIBA-GEIGY, polyacrylic acids of the SYNTHALEN K type, and the salts, in particular of sodium, of polyacrylic acid (corresponding to the INCI name sodium acrylate copolymer) and more particularly a crosslinked sodium polyacrylate (corresponding to the INCI name sodium acrylate copolymer (and) caprylic/capric triglyceride) sold under the name “LUVIGEL EM” by the company,
the copolymers of acrylic acid and acrylamide sold in the form of their sodium salt under the names “RETEN” by the company HERCULES, sodium polymethacrylate sold under the name “DARVAN N^{o} 7” by the company VANDERBILT, the sodium salts of polyhydroxycarboxylic acids sold under the name “HYDAGEN F” by the company HENKEL,
polyacrylic acid/alkyl acrylate copolymers of the PEMULEN type,
AMPS (polyacrylamidomethylpropanesulphonic acid partially neutralized with aqueous ammonia and highly crosslinked) marketed by the company CLARIANT,
AMPS derivatives such as
   the AMPS/acrylamide copolymers of the SEPIGEL or SIMULGEL type marketed by the company SEPPIC,
   the AMPS/polyoxyethylenated alkyl methacrylate copolymers (crosslinked or not) of the ARISTOFLEX HMS type marketed by the company CLARIANT,
   and mixtures thereof,
proteins such as proteins of plant origin such as wheat or soybean proteins; proteins of animal origin such as keratins, for example keratin hydrolysates and sulphonic keratins;
anionic, cationic, amphoteric or nonionic chitin or chitosan polymers;
cellulose polymers and their derivatives such as hydroxyethylcellulose, hydroxypropylcellulose, methyl-cellulose, ethylhydroxyethylcellulose, carboxymethyl-cellulose, and the quaternized derivatives of cellulose;
vinyl polymers, such as polyvinylpyrrolidones, copolymers of methyl vinyl ether and malic anhydride, copolymer of vinyl acetate and crotonic acid, copolymers of vinylpyrrolidone and vinyl acetate; copolymers of vinylpyrrolidone and caprolactam; polyvinyl alcohol;
polymers of natural origin, optionally modified, such as:
   gum arabic, guar gum, xanthan derivatives, karaya gum;
   alginates and carrageenans;
   glycoaminoglycans, hyaluronic acid and its derivatives;
   shellac resin, sandarac gum, dammars, elemis, copals;
   deoxyribonucleic acid;
   mucopolysaccharides such as hyaluronic acid, chondroitinsulphate, and mixtures thereof.

In particular, the composition is free of silicas.

Exothermic Metal Salts

The metal salts used in the present invention are salts having exothermic properties, that is to say metal salts which release heat when they are moistened. They are preferably chosen in particular from alkaline-earth metal salts, magnesium salts and mixtures thereof.

As alkaline-earth metal salts, there may be mentioned in particular calcium salts and, more especially, calcium halides such as calcium iodide, chloride and bromide. Calcium chloride is preferably used.

As magnesium salts, magnesium sulphate may be mentioned in particular.

It is also possible to use a mixture of several salts.

According to a preferred embodiment of the invention, the exothermic metal salt is chosen from magnesium sulphate, calcium chloride and mixtures thereof.

The quantity of metal salt(s) may range for example from 0.1 to 50% by weight, preferably from 5 to 40% by weight and even better from 10 to 30% by weight relative to the total weight of the composition, including for example 7, 12, 15, 18, 20, 25, 28, 33, 38, 40, 44, and 48% including all values and subranges between stated values.

Polyols

According to a preferred embodiment of the invention, the composition of the invention additionally contains at least one polyol. Without being bound by theory, it is believed that the polyols supplement the exothermic action of the salts.

As polyols, there may be mentioned in particular the polyols having at least 2 hydroxyl groups and at least 3 carbon atoms, such as glycerine, diglycerine, and the glycols such as propylene glycol, dipropylene glycol, butylene glycol, hexylene glycol, polyethylene glycol, and polyethylene glycols having a molecular weight of less than 600, such as PEG-8 (or polyethylene 400), the sugars such as sorbitol, and mixtures thereof.

As polyols, glycerine, butylene glycol, propylene glycol, dipropylene glycol, PEG-8 and mixtures thereof are preferably used.

The quantity of polyol(s) depends of the quantity of salts present in the composition and the heating effect desired. It may range for example from 0.5 to 80% by weight, preferably from 5 to 75% by weight, even better from 10 to 70% by weight and better still from 20 to 65% by weight and better still from 30 to 60% by weight relative to the total weight of the composition.

Anhydrous Medium

The composition according to the invention preferably comprises an oily medium comprising at least one oil. The quantity of oil(s) may range for example from 5 to 80% by weight, preferably from 10 to 60% by weight, even better from 10 to 40% by weight relative to the total weight of the composition.

The expression “oil” is understood to mean a fatty substance that is liquid at room temperature (25° C.).

As oils which can be used in the composition of the invention, there may be mentioned for example:
hydrocarbon oils of plant origin, such as liquid triglycerides of fatty acids comprising from 4 to 10 carbon atoms such as the triglycerides of heptanoic or octanoic acids or alternatively, for example, sweet almond oil, sunflower oil, maize oil, soybean oil, gourd oil, coriander oil, grape seed oil, sesame oil, hazelnut oil, apricot oil (Prunus Armenica oil), macadamia oil, arara oil, oil from rUniqeman, avocado oil, the triglycerides of caprylic/capric acids such as those sold by the company Stearineries Dubois or those sold under the names Miglyol 810, 812 and 818 by the company Dynamit Nobel, jojoba oil, shea butter oil;
synthetic esters and ethers, in particular of fatty acids, such as the oils of formulae R¹COOR² and R¹OR² in which R¹ represents the residue of a fatty acid comprising from 8 to 29 carbon atoms, and R² represents a branched or unbranched hydrocarbon chain containing from 3 to 30 carbon atoms, such as for example Purcellin oil or 660084 PCL-LIQUID from the company SYMRISE (mixture of cetylstearyl 2-ethylhexanoate and isopropyl myristate), isononyl isononanoate, isopropyl myristate, isopropyl palmitate, 2-ethylhexyl palmitate (or octyl palmitate), 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, heptanoates, octanoates and decanoates of fatty alcohols; polyol esters, such as propylene glycol dioctanoate, neopentyl glycol diheptanoate and diethylene glycol diisononanoate; and the pentaerythritol esters such as pentaerythrityl tetraisostearate;
linear or branched hydrocarbons, of mineral or synthetic origin, such as volatile or non-volatile paraffin oils, and derivatives thereof, petroleum jelly, polydecenes, hydrogenated polyisobutene such as Parleam® oil;
fatty alcohols having from 8 to 26 carbon atoms, such as cetyl alcohol, stearyl alcohol and a mixture thereof (cetearyl alcohol), octyldodecanol, 2-butyloctanol, 2-hexyldecanol, 2-undecylpentadecanol, oleyl alcohol or linoleyl alcohol;
partially hydrocarbon-based and/or silicone-based fluorinated oils such as those described in the document JP-A-2-295912. As fluorinated oils, there may also be mentioned perfluoromethylcyclopentane and perfluoro-1,3-dimethylcyclohexane, sold under the names “FLUTEC PC1®” and “FLUTEC PC3®” by the company BNFL Fluorochemicals; perfluoro-1,2-dimethylcyclobutane; perfluoroalkanes such as dodecafluoropentane and tetradecafluorohexane, sold under the names “PF 5050®” and “PF 5060®” by the company 3M, or alternatively bromoperfluorooctyl sold under the name “FORALKYL®” by the company Atochem; nonafluoromethoxybutane sold under the name “MSX 4518” by the company 3M and nonafluoroethoxyisobutane; perfluoromorpholine derivatives, such as 4-trifluoromethyl-perfluoro-morpholine sold under the name “PF 5052®” by the company 3M;
silicone oils such as volatile or non-volatile polymethylsiloxanes (PDMS) containing a linear or cyclic silicone chain, which are liquid or pasty at room temperature, in particular cyclopolydimethyl-siloxanes (cyclomethicones) such as cyclohexasiloxane and cyclopentasiloxane; polydimethylsiloxanes comprising alkyl, alkoxy or phenyl groups which are pendant or at the end of the silicone chain, groups having from 2 to 24 carbon atoms; phenylated silicones such as phenyltrimethicones, phenyldimethicones, phenyltrimethylsiloxydiphenylsiloxanes, diphenyl-dimethicones, diphenylmethyldiphenyl trisiloxanes, 2-phenylethyltrimethylsiloxysilicates, and polymethyl-phenylsiloxanes;
mixtures thereof.

The expression “hydrocarbon oil” is understood to mean, in the list of oils mentioned above, any oil predominantly containing carbon and hydrogen atoms, and optionally ester, ether, fluorinated, carboxylic acid and/or alcohol groups.

When the composition of the invention is used as a makeup-removing composition, it preferably contains at least one makeup-removing oil, to which one or more other oils may be added which may or may not be makeup-removing oils. The makeup-removing oils may be chosen in particular from branched hydrocarbons of mineral origin such as hydrogenated polyisobutene, the fatty acid esters described above, and mixtures thereof. As fatty acid esters which can be used as makeup-removing oils, there may be mentioned more particularly ethylhexyl palmitate, ethylhexyl stearate, isopropyl myristate, isopropyl palmitate, isobutyl palmitate, pentaerythrityl caprate/caprylate, cetearyl isononanoate, isodecyl isononanoate, isononyl isononanoate, isotridecyl isononanoate, 2-ethylhexyl caprate/caprylate, and mixtures thereof.

In addition to the oils indicated above, the composition of the invention may contain other fatty substances, such as fatty acids containing from 8 to 30 carbon atoms, such as stearic acid; silicone resins such as trifluoromethyl-C1-4-alkyldimethicone and trifluoropropyldimethicone; silicone gums (INCI name: Dimethiconol) alone or as a mixture with a silicone oil, such as the product marketed by the company Dow Corning under the name Dow Corning 1501 Fluid, which is a mixture of Dimethiconol and Cyclopentasiloxane in the ratio 14.7/85.3 (dimethiconol); silicone elastomers such as those marketed under the names KSG by the company Shin-Etsu; waxes, for example mineral waxes, waxes of animal origin such as beeswax, waxes of plant origin, hydrogenated oils that are concrete at 25° C., fatty esters and glycerides that are concrete at 25° C., synthetic waxes such as polymethylene wax, silicone waxes; and mixtures thereof.

Additives

The composition may further contain one or more other additives, in particular lipophilic additives, including those which are conventionally used in cosmetic compositions.

There may be mentioned in particular exothermic compounds other than salts and polyols, such as for example zeolites which are silicoaluminates. As zeolites, there may be mentioned in particular activated zeolites, and for example the zeolites A, the zeolites X such as those marketed by the companies Fluka and Union Carbide, the zeolites MAP as described in the document EP-A-384070, the activated zeolites A as described in the document EP-A-187912 and in particular those marketed under the name ADVERA 401N ET ADVERA 402N by the company PQ Corporation.

As additives, mention may also be made of perfumes; preservatives; antioxidants; sequestrants; fillers; colorants, cosmetic or dermatological active agents, or mixtures thereof. These adjuvants are used in the usual proportions for cleansing and/or care compositions, and for example from 0.01 to 10% of the total weight of the composition. These adjuvants should be of a nature such that and used in quantities such that they do not disrupt the properties sought for the composition of the invention.

According to a particular embodiment of the invention, the composition according to the invention contains, as fillers, exfoliating particles which will allow scrubbing of the skin. As exfoliating particles, use may be made of the exfoliating or scrubbing particles of mineral, plant or organic origins. Thus, it is possible to use for example polyethylene beads or powder, such as those marketed under the name Microthene MN 727 or Microthene MN 710-20 by the company Equistar or such as the powder marketed under the name Gotalene 120 Incolore 2 by the company Dupont; nylon particles such as those marketed by the company Arkema under the name Orgasol 2002 EXD NAT COS; fibres such as polyamide fibres, such as those marketed by the company Utexbel under the name PULPE POLYAMIDE 12185 TAILLE 0, 3 MM, polyvinylchloride powder; pumice stone (INCI name: pumice) such as pumice 3/B from Eyraud; the ground shells of fruit kernels such as the ground products of apricot kernels or of walnut shells; sawdust; glass beads; alumina (aluminium oxide) (INCI name: Alumina) such as the product marketed under the name Dermagrain 900 by the company Marketech International, sugar crystals; beads which melt during application to the skin, such as for example the spheres based on mannitol and cellulose which are marketed under the names Unispheres by the company Induchem, capsules based on agar which are marketed under the names Primasponge by the company Cognis, and the spheres based on jojoba esters which are marketed under the names Floraspheres by the company Floratech; and mixtures thereof.

The composition according to the invention may also contain other fillers such as, for example, talc, modified or unmodified starch, and in particular starches esterified with octenylsuccinic anhydride and more particularly “Aluminium Starch octenyl succinate” such as the product marketed by the company National Starch under the name Dry-Flo.

These fillers (exfoliants and the like) may be present in a quantity ranging for example from 0.5 to 20% by weight, preferably from 1 to 15% by weight, even better from 1 to 10% and better still from 2 to 5% by weight relative to the total weight of the composition.

According to another particular embodiment, the composition according to the invention is free of fillers or exfoliating particles as described above.

As active agents, there may be mentioned any care or cleansing active agent normally used in the cosmetic field, and in particular antibacterial agents such as octopirox and triclosan, keratolytic agents such as salicylic acid, lactic acid or glycolic acid, essential oils, vitamins such as vitamin C (ascorbic acid), vitamin A (retinol), vitamin PP (niacinamide), vitamin B3 (panthenol) and their derivatives.

The composition of the invention is particularly suitable for cleansing and/or removing makeup from the skin and/or the mucous membranes and more particularly for cleansing and/or removing makeup from and/or scrubbing the skin.

A subject of the invention is also the cosmetic use of the composition as defined above, for cleansing and/or removing makeup from and/or scrubbing the skin.

The composition of the invention is generally preferably provided in the form of a gel, but other forms may be used. During use, the composition is applied to the skin (in particular the skin of the face), preferably as a thick layer, and then the hands are moistened and the face is massaged with the wet hands. It is possible to optionally wet the hands again and to again massage the face. The face is finally rinsed. Another method includes first wetting the skin, and then in applying the composition to the wet skin and massaging the face, after having wetted the hands or otherwise.

Accordingly, a subject of the invention is also a method for removing makeup from and/or cleansing and/or scrubbing the skin, comprising wetting the skin, in applying to the skin the composition as defined above, in massaging and in rinsing the skin.

A subject of the invention is also a method for removing makeup from and/or cleansing the skin, comprising applying to the skin the composition as defined above, in wetting the hands, in massaging the face with the wet hands, and in rinsing the skin.

The composition of the invention may be adapted for shaving the skin for example of the face or of the legs.

Consequently according to one aspect, a subject of the invention is also a method of shaving, comprising applying to the surface of the skin to be shaved a composition as defined in any one of the preceding claims and then in shaving the body hair by means of a razor.

The example below is given by way of nonlimiting illustration in order to better demonstrate the characteristics of the invention. The quantities are given therein in % by weight.

### Example 1

### Foaming Heating Composition

| | |
|---|---|
| PEG-8 | qs 100% |
| Glycerine | 20% |
| Magnesium sulphate and water mixture (98/2) | 20% |
| Citric acid | 0.5% |
| Cocoamidopropylbetaine | 5% |
| Sodium lauryl sulphate (EMPICOL TL40 FL from | 5% |
| Huntsman) | |

The above written description of the invention provides a manner and process of making and using it such that any person skilled in this art is enabled to make and use the same, this enablement being provided in particular for the subject matter of the appended claims, which make up a part of the original description.

The phrases “selected from the group consisting of,” “chosen from,” and the like include mixtures of the specified materials. Terms such as “contain(s)” and the like are open terms meaning ‘including at least’ unless otherwise specifically noted. The term “mentioned” notes exemplary embodiments, and is not limiting to certain species.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition comprising, in an anhydrous medium, an exothermic metal salt, an amphoteric foaming surfactant and an anionic foaming surfactant, in an anionic surfactant/amphoteric surfactant ratio of 0.5 to 2, the composition comprising less than 1% of gelling agent based on total weight of the composition.

2. The composition according to claim 1, wherein the exothermic metal salt is chosen from alkaline-earth metal salts, magnesium salts, and mixtures thereof.

3. The composition according to claim 1, wherein the exothermic metal salt is chosen from magnesium sulphate, calcium chloride, and mixtures thereof.

4. The composition according to claim 1, wherein the quantity of exothermic metal salt(s) ranges from 0.1 to 50% by weight relative to the total weight of the composition.

5. The composition according to claim 1, wherein the amphoteric surfactant is chosen from N-alkylamidobetaines and their derivatives.

6. The composition according to claim 1, wherein the amphoteric surfactant is cocamidopropylbetaine.

7. The composition according to claim 1, wherein the anionic surfactant is chosen from alkyl sulphates and their salts.

8. The composition according to claim 1, wherein the anionic surfactant is sodium lauryl sulphate.

9. The composition according to claim 1, wherein the amphoteric surfactant and the anionic surfactant are present in the composition in an anionic surfactant/amphoteric surfactant ratio of 0.7 to 1.5.

11. The composition according to claim 1, wherein the amphoteric foaming surfactant and anionic foaming surfactant are present in a total amount of 0.1 to 30% by weight relative to the total weight of the composition.

12. The composition according to claim 1, wherein the amphoteric foaming surfactant and anionic foaming surfactant are present in a total amount of 2 to 10% by weight relative to the total weight of the composition.

13. The composition according to claim 1, wherein the composition is free of silica.

14. The composition according to claim 1, wherein the composition is in the form of a gel.

15. The composition according to claim 1, wherein the composition comprises less than 0.5% of gelling agent based on total weight of the composition.

16. A method for removing makeup from and/or cleansing and/or scrubbing the skin, comprising wetting the skin, applying to the skin the composition according to claim 1, massaging and rinsing the skin.

17. A method of shaving, comprising applying to the surface of the skin to be shaved a composition according to claim 1 and then shaving body hair from the surface of the skin to be shaved.
